(19) United States
(12) Patent Application Publication
(10) Pub. No.: US 2026/0248789 A1
(43) Pub. Date: Aug. 27, 2026

(54) CONTROLLED RELEASE PDE10A FORMULATIONS

(71) Applicants: Merck Sharp & Dohme LLC, Rahway, NJ (US); Merck Sharp & Dohme (UK) Limited, Hoddesdon, Hertfordshire (GB)

(72) Inventors: James C. Dinunzio, Flemington, NJ (US); David Harris, New Providence, NJ (US); Maria Sharlini Kumar, London (GB); Michael John Pollitt, St. Andrews (GB); Jovana Radojevic, Harrison, NJ (US); Graciela Terife, Jersey City, NJ (US)

(73) Assignees: Merck Sharp & Dohme LLC, Rahway, NJ (US); Merck Sharp & Dohme (UK) Limited, Hoddesdon, Hertfordshire (GB)

(21) Appl. No.: 19/163,501

(22) PCT Filed: Mar. 25, 2024
(86) PCT No.: PCT/US2024/021265

§ 371 Date: Sep. 9, 2025

Related U.S. Application Data
(60) Provisional application No. 63/455,403, filed on Mar. 29, 2023.

(51) Int. Cl. A61K 31/506; A61K 9/20

(52) U.S. Cl. CPC A61K 31/506; A61K 9/2009; A61K 9/2013; A61K 9/2018; A61K 9/2031; A61K 9/2054; A61K 9/2086

(57) ABSTRACT

The present disclosure relates generally to treatment of central nervous system disorders associated with phosphodiesterase 10A (PDE10A) such as schizophrenia, bipolar disease, and Alzheimer's disease, as therapeutics for neurological and psychiatric disorders. This disclosure provides controlled-release formulations of 2-methyl-N-((5-methyl-1,3,4-thiadiazol-2-yl)methyl)-6-(((1S,2S)-2-(5-methylpyridin-2-yl)cyclopropyl)methoxy)pyrimidin-4-amine (Compound A) and their use in the treatment of schizophrenia and other psychiatric disorders that improves the tolerability profile.

### CROSS-REFERENCE TO RELATED APPLICATIONS

This application claims the benefit of priority to U.S. Provisional Application No. 63/455,403, filed Mar. 29, 2023, the disclosure of which is incorporated herein by its entirety.

### BACKGROUND OF THE INVENTION

The present disclosure relates generally to treatment of central nervous system disorders associated with phosphodiesterase 10A (PDE10A) such as schizophrenia, bipolar disease, and neuropsychiatric symptoms associated with Alzheimer's disease. Inhibition of PDE10A is believed to be useful in the treatment of schizophrenia and a wide variety of conditions or disorders that would benefit from increasing levels of cAMP and/or cGMP within neurons, including a variety neurological, psychotic, anxiety and/or movement disorders.

Therapeutic compounds, with a short elimination half-life, that are released quickly in the gastrointestinal tract, are often characterized by initial sharp increases in plasma concentrations followed by a steep decline as they undergo elimination. The “peak-to-trough” plasma concentration ratio (PTR), defined as the ratio of the maximum/peak plasma concentration (Cmax) and the trough/minimum plasma concentration (Cmin) over a dosing interval (e.g., once-daily), is useful for representing how quickly it may take for a patients drug concentrations to decline from their maximal concentration to pre-dose levels. Compounds that exhibit a high PTR may not provide the needed sustained target occupancy over the preferred dosing interval and require either administration of a higher daily dose and/or increased frequency of administration.

Controlled-release (CR) formulations have been prepared in a number of ways, generally to slow or delay the delivery of the active ingredient to the site of absorption. The compositions of the present invention solve the difficult problem of providing therapeutically effective sustained plasma concentration of a PDE10A inhibitor by controlling release of the inhibitor using formulations with zero-order release profiles extending for over 12 hours after ingestion.

Several potent PDE10A inhibitors have been described, see for example, U.S. Pat. No. 9,062,059 (incorporated herein in its entirety), U.S. Pat. Nos. 8,957,077, 8,975,261, 9,359,348, 9,376,450 and EP2,776,418. However, clinical trials have shown that without optimization of pharmacokinetic profiles and dose, the benefit of PDE10A inhibitors in the treatment of schizophrenia is confounded by insufficient target engagement and side effects. Understanding of the requirements for PDE10A inhibitors constitutes a challenging but promising field of drug discovery and development.

There remains a need for CR formulations of PDE10A inhibitors with an improved tolerability profile (reduced adverse events/side effects, e.g., dystonia, akathisia) and patient compliance.

### SUMMARY OF THE INVENTION

U.S. Pat. No. 9,062,059 discloses potent PDE10A inhibitors, including 2-methyl-N-((5-methyl-1,3,4-thiadiazol-2-yl)methyl)-6-(((1S,2S)-2-(5-methylpyridin-2-yl)cyclopropyl)methoxy)pyrimidin-4-amine (Compound A), an ester derivative, geometric isomer, stereoisomer, or optical isomer thereof as therapeutics for neurological and psychiatric disorders. This disclosure provides CR formulations of Compound A and their use in the treatment of schizophrenia and other psychiatric disorders that improves the tolerability profile. An aspect of this disclosure provides CR formulations of Compound A and their use in the treatment of schizophrenia and other psychiatric disorders that allows high peak concentrations (Cmax) with minimal or no incidence of dystonia. Another aspect of this disclosure provides CR formulations of Compound A wherein the excipients of the formulation are not dose dependent. Another aspect of this disclosure provides CR formulations of Compound A and their use in the treatment of schizophrenia and other psychiatric disorders that can be dosed at high concentrations without titration. Another aspect of this disclosure provides CR formulations of Compound A and their use in the treatment of schizophrenia and other psychiatric disorders that can be administered at doses that provide high peak concentrations (Cmax) with minimal or no incidence of dystonia. Another aspect of this disclosure provides controlled-release formulations of Compound A that provide high peak concentrations (Cmax) and their use in the treatment of schizophrenia and other psychiatric disorders.

This disclosure further provides CR formulations of Compound A and their use in the treatment of schizophrenia and other psychiatric disorders wherein the incidence of dystonia is reduced or eliminated. This disclosure further provides CR formulations of Compound A and their use in the treatment of schizophrenia and other psychiatric disorders wherein the incidence of dystonia is reduced or eliminated when administering escalated doses (16 mg or higher) to ensure effective concentrations can be maintained for the duration of the dosing interval. The disclosure further provides CR formulations of Compound A having at or near zero-order release profiles, with pharmacokinetic profiles exhibiting minimal fluctuation between peak concentrations and trough concentrations which improves the tolerability profile. An aspect of this embodiment is CR formulations of Compound A with PTR from about 1.0 to about 3.5. An aspect of this disclosure is realized when PTR is from about 1.0 to about 2.0. Another aspect of this disclosure is realized when the peak concentration to trough concentration ratio is from about 1.0 to about 1.5. Another aspect of this disclosure is realized when the peak concentration to trough concentration ratio is from about 1.0 to about 1.3.

Another aspect of the disclosure is realized wherein patients are dosed with about 20 mg to about 80 mg, about 17 to about 47 mg, 20 mg to about 36 mg, or about 17 mg to about 24 mg of CR formulations of Compound A with minimal or no dystonia. An embodiment of this aspect is realized when the CR formulation of Compound A is compared to an immediate-release (IR) formulation of Compound A having the same dose.

Another aspect of the disclosure is realized wherein dystonia is healthy participants and/or patients suffering with schizophrenia or other psychiatric disorders are dosed with CR formulations described herein of about 17 mg to about 47 mg, about 20 mg to about 36, or about 20 mg to about 24 mg. An embodiment of this aspect is realized when the CR formulation of Compound A is compared to an IR formulation of Compound A having the same dose.

Another aspect of the disclosure is realized wherein healthy participants and patients suffering with schizophrenia or other psychiatric disorders are dosed with CR formulations described herein of about 20 mg to about 36, about 20 mg to about 24 mg, or 24 mg of the CR formulations of Compound A without titration wherein patients experience minimal or no observed dystonia. An embodiment of this aspect is realized when the CR formulation of Compound A is compared to an IR formulation of Compound A having the same dose.

An aspect of the disclosure are CR formulations comprising about 2 mg to about 80 mg of Compound A dosed once daily. Another aspect of the disclosure are CR formulations comprising from about 16 mg to about 36, about 20 mg to about 24 mg, or 24 mg of Compound A without titration. An aspect of this embodiment is realized when the release of Compound A is controlled and occurs over a period of about 12-24 hours. After repeat administration of the composition into an in vivo environment, steady-state Cmax plasma concentrations are achieved by ~10 to 24 hours and are generally sustained over the dosing interval. Another aspect of this of this embodiment is realized when Compound A is dosed once daily with about 17 mg to about 47 mg, about 20 mg to about 36 mg or about 20 mg to about 24 mg, wherein the incidence of dystonia is mitigated. Another aspect of this embodiment is realized when the CR formulation of Compound A is administered once daily without incidence of dystonia. Another aspect of this embodiment is realized when the CR formulation of Compound A is administered at about 20 mg to about 47 mg once daily, without incidence of dystonia.

Another aspect of the disclosure is CR formulations wherein the amount of Compound A present in said formulation is estimated to produce generally sustained/constant enzyme occupancy (EO) levels at both the peak and trough of the 24 hr dosing interval (Table 2-5) in contrast to the IR formulation (Table 1). A subembodiment of this aspect of the disclosure is realized when high levels of the estimated EO are sustained at both peak and trough at doses ranging from about 17 mg to about 47 mg, about 20 mg to about 36 mg, or about 20 mg to 24 mg. An aspect of this subembodiment is realized when the estimated EO levels at both peak and trough are sustained above 75% at steady state. Another aspect of this subembodiment is realized when the estimated EO levels at both peak and trough are sustained from about 65%-99%. 67%-99%, 70%-99%, 80-99%, 85%-99% at steady state. Another aspect of this subembodiment is realized when the estimated EO levels at both peak and trough are sustained from about 70% to 99% at doses ranging from about 17 mg to 47 mg, or about 20 mg to 36 mg in both the healthy population and population suffering from schizophrenia with no reported events of dystonia. Another aspect of this subembodiment is realized when the estimated EO levels at both peak and trough are sustained from about 70% to 99% at doses ranging from about 17 mg to 47 mg or 20 mg to 36 mg in patients suffering from schizophrenia with no reported events of dystonia. Another aspect of this subembodiment is realized when the estimated EO levels are sustained from about 70% to 99% at doses ranging from about 17 mg to 47 mg or about 20 mg to 36 mg in healthy population with no reported events of dystonia.

Other aspects of the invention can be realized upon review of the specification as a whole.

### BRIEF DESCRIPTION OF THE DRAWINGS

FIG. 1 Illustrates PDE10A Enzyme Occupancy Versus Compound A Plasma Concentrations Following Single Oral Dose Administration of Compound to Healthy Subjects

FIG. 2 Illustrates that the Tmax for the IR formulation of Compound A ranged from 0.5 to 2 h and PTR of about 12-17.

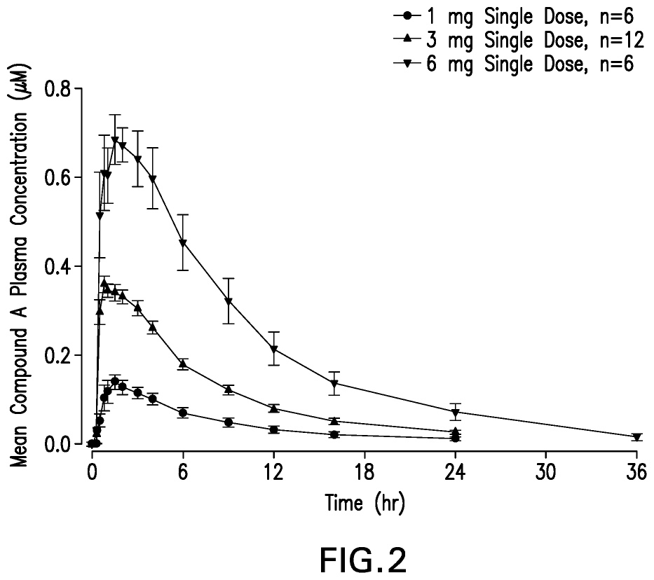

FIG. 3 Illustrates the in-vitro dissolution profiles of CR formulations of Compound A designed to achieve 80% release over periods of 12, 16 and 20 hours, respectively.

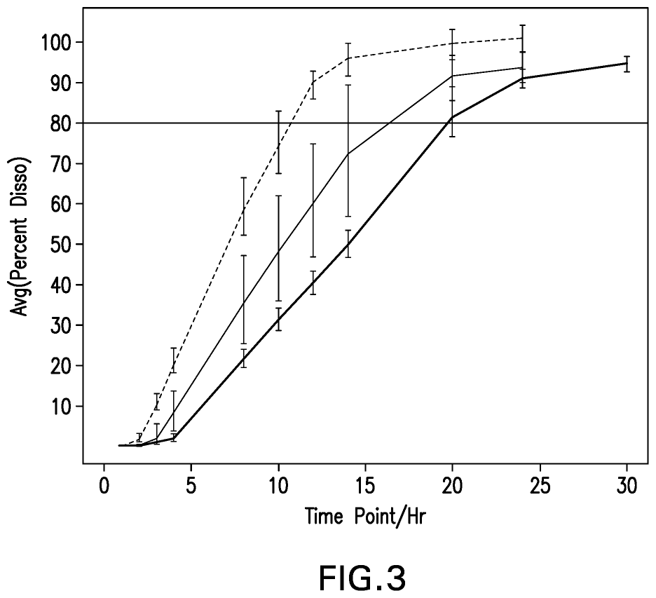

FIG. 4 Illustrates the pharmacokinetic profiles of CR formulations exhibiting 80% release over periods of 12, 16 and 20 hours.

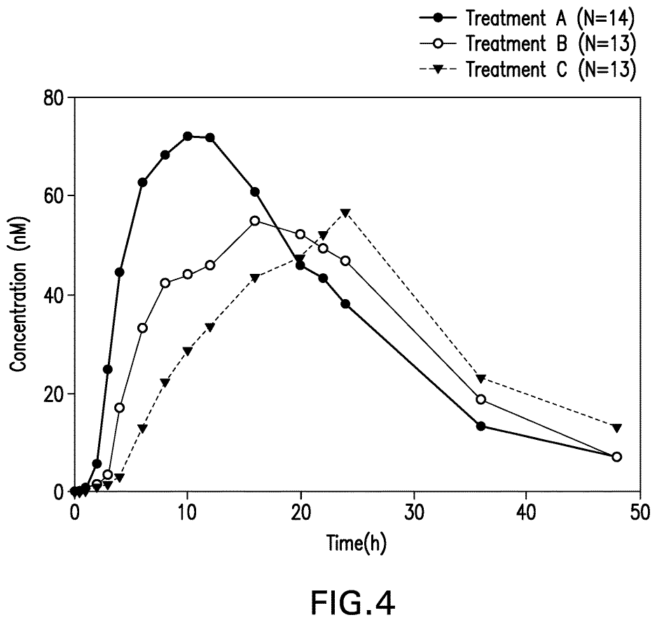

Various aspects described herein are described in further detail in the following subsections.

### DETAILED DESCRIPTION OF THE INVENTION

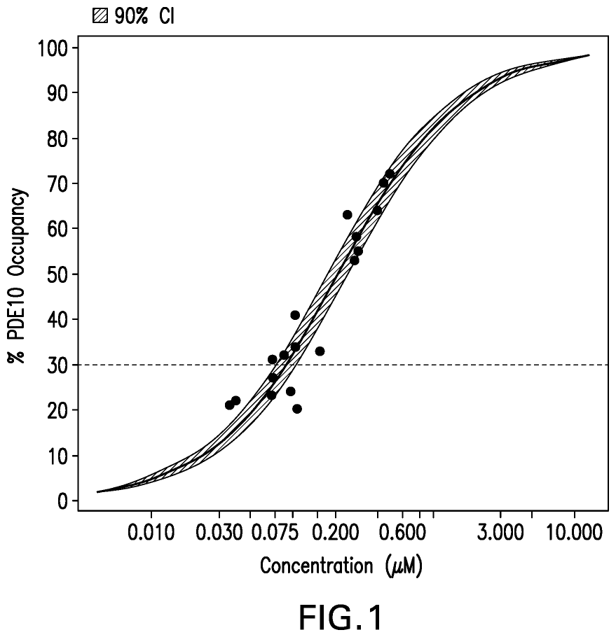

Studies with Compound A and other PDE10A inhibitors have shown activity in assays supporting clinical efficacy in the treatment of schizophrenia at enzyme occupancies (EO) >30% (Smith et al., Biological Psychiatry 2022, Volume 91, Issue 9, S309-S310 and Li, Y W, et al., Neuropharmacology. 2016 March; 102:121-35. doi: 10.1016/j.neuropharm.2015.10.037. Epub 2015 Oct. 30. PMID: 26522433). Data from a rising single-dose study and a PET study with an IR formulation of Compound A were used to build an EO-concentration relationship (FIG. 1). These studies in healthy participants demonstrated that administration of Compound A IR (3 mg and 6 mg) achieved estimated EO values at Cmax of ~64% and ~78%, respectively (Table 1), and rapidly cleared from the brain in a few hours (estimated EO values at C24 of ~8.9% and ~21%). However, dystonia was reported and appeared to be related to the time of maximum concentration (Tmax).

This disclosure provides CR tablet formulations of Compound A wherein overall psychiatric tolerability limitations such as dystonia, akathisia, anxiety, depression, nausea, etc., are either mitigated and/or eliminated. Particularly, the CR formulations of Compound A can be administered once daily at high clinical doses (e.g., 20 mg, 24 mg, 36 mg, etc.) without dystonia. Specifically, the present disclosure provides a CR tablet formulation comprising a
1) first layer having about 1%-25% by weight of Compound A:

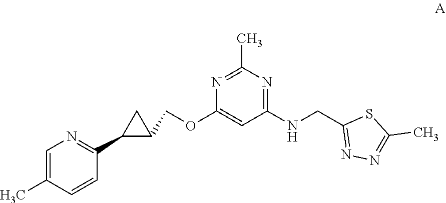

or a pharmaceutically acceptable salt, ester derivative, geometric isomer, stereoisomer, or optical isomer thereof and about 60-90% by weight low viscosity polyethylene oxide (PEO) polymer;
2) second layer comprising about 50%-70% by weight high viscosity polyethylene oxide polymer, about 10%-40% by weight metal halide and about 5%-30% by weight tableting agent and;
3) a semipermeable coating wherein the coating comprises about 1%-15% by weight of the tablet, said tablet having an orifice in the first layer.

An embodiment of this disclosure is realized wherein the percent of Compound A and low viscosity PEO is relative to the total first layer weight and the percent of high viscosity PEO, metal halide and tableting agent is relative to the total second layer weight.

In an embodiment, Compound A, or its ester derivatives, geometric isomers, stereoisomers, or optical isomers thereof is present about 4%-25% by weight of the total first layer weight, preferably about 5%-15% by weight.

In another embodiment, low viscosity polyethylene oxide (PEO) is any low viscosity PEO with a molecular weight range (g/mol) of about 100,000 to 300,000; preferably 200,000 g/mol. A subembodiment of this aspect is realized when low viscosity PEO is present about 65%-88% by weight of the total first layer weight, preferably about 70%-88% by weight.

In another embodiment, high viscosity polyethylene oxide (PEO) is any high viscosity PEO with a molecular weight range (g/mol) of about 4,000,000 to 7,000,000 g/mol, preferably about 5,000,000 g/mol. A subembodiment of this aspect is realized when high viscosity PEO is present about 50%-70% by weight of the total second layer weight, preferably about 60%-68% by weight.

Tableting agent is any agent known in the art used for the long term stabilization of drugs, bulking of solid dose formulations, and enhancing the therapeutic effects of medications. In an embodiment a tableting agent selected from lactose, spray dried lactose, microcrystalline cellulose (e.g., Avicel PH101 and PH102), Mannitol (e.g., Pearlitol SD200), sorbitol, dibasic calcium phosphate dehydrate, calcium sulphate dehydrate and the like, preferably microcrystalline cellulose is optionally present in the second layer. A subembodiment of this aspect is realized when tableting agent is present about 0%-25% by weight of the total second layer weight, preferably about 10%-20% by weight.

In another embodiment, the metal halide is an osmotic agent selected from sodium chloride, potassium chloride, calcium chloride, potassium iodide, and the like, preferably sodium chloride, wherein the sodium chloride is present as a powder. A subembodiment of this aspect is realized when the metal halide is present about 10%-40% by weight of the total second layer weight, preferably about 15%-25% by weight.

Typical semipermeable polymers, known to the art as osmosis and reverse osmosis membranes, are cellulose acylate, cellulose diacylate, cellulose triacylate, cellulose acetate, cellulose diacetate, cellulose triacetate, agar acetate, amylose triacetate, beta glucan acetate, acetaldehyde dimethyl acetate, cellulose acetate ethyl carbamate, polyamides, polyurethanes, sulfonated polystyrenes, cellulose acetate phthalate, cellulose acetate methyl carbamate, cellulose acetate succinate, cellulose acetate, dimethylaminoacetate, cellulose acetate ethyl carbamate, cellulose acetate chloroacetate, cellulose dipalmatate, cellulose dioctanoate, cellulose dicap rylate, cellulose dipentanlate, cellulose acetate valerate, cellulose acetate, succinate, cellulose propionate succi-nate, methyl cellulose, cellulose acetate p-toluene sulfo-nate, cellulose acetate butyrate, cross-linked selectively semipermeable polymers formed by the coprecipitation of a polyanion and a polycation as disclosed in U.S. Pat. Nos. 4,327,725; 3,173,876; 3,276,586; 3,541,005; 3,541,006; and 3,546,142, semipermeable polymers as disclosed by Loeb and Sourirajan in U.S. Pat. No. 3,133,132, lightly cross-linked polystyrene derivatives, cross-linked poly(-sodium styrene sulfonate), poly(vinylbenzyltrimethyl ammonium chloride), cellulose acetate having a degree of substitution up to 1 and an acetyl content up to 21%, cellulose diacetate having a degree of substitution of 1 to 2 and an acetyl content of 21 to 35%, cellulose triacetate havin a degree of substitution of 2 to 3 and an acetyl content of 35 to 44%, as disclosed in U.S. Pat. No. 4,160,020.

In an embodiment, semipermeable coating is selected from the group comprising cellulose acetate, ethyl cellulose, or a combination thereof. A subembodiment is realized when semipermeable coating comprises a mixture of cellulose acetate, ethyl cellulose, or mixture thereof and low molecular weight polyethylene glycol. In an aspect of this subembodiment, the 1%-15%, preferably 5%-15%, by weight of the core tablet of semipermeable coating comprises cellulose acetate, ethyl cellulose, or mixture thereof, with about 5%-20%, preferably 5%-10%, by weight of the coating of low molecular weight polyethylene glycol. In an aspect of this embodiment, polyethylene glycol has a molecular weight range from about 600-10,000 g/mol, preferably about 3000 to about 4000 g/mol, more preferably about 3350 g/mol.

In another embodiment, the orifice is about 0.1 to 1 mm diameter.

In an embodiment of the CR tablet formulation, Compound A, or a pharmaceutically acceptable salt, ester derivative, geometric isomer, stereoisomer, or optical isomer thereof is present at about 4%-25% by weight of the total first layer weight, low viscosity polyethylene oxide (PEO) has a molecular weight range (g/mol) of about 100,000 to 300,000; and is present at about 65%-88% by weight of the total first layer weight, high viscosity polyethylene oxide (PEO) has a molecular weight range (g/mol) of about 4,000,000 to 7,000,000 g/mol and is present 60%-68% by weight of the total second layer weight. A subembodiment of this aspect is realized when tableting agent is not present. A subembodiment of this aspect is realized when tableting agent is present up to about 25% by weight of the total second layer weight, preferably about 10%-20% by weight. Another subembodiment of this aspect is realized when metal halide is present at about 15%-25% by weight of the total second layer weight, preferably 15%-20% by weight. Another subembodiment of this aspect is realized when 5%-15% by weight of the tablet core of semipermeable coating is present and comprises cellulose acetate, ethyl cellulose, or mixture thereof, containing about 5%-20%, preferably 5%-10%, by weight of the coating of low molecular weight polyethylene glycol. It should be noted that the tablet composition percentages are based on total weight of each layer separately, where total weight for each layer is 200 mg in Example 2 and 100 mg in Example 3.

In an embodiment of the CR tablet formulation, compositions containing a ratio of low viscosity PEO to high viscosity PEO of about 1:0.5, about 1:0.7, about 1:0.8, about 1:0.9, about 1:1, about 1:1.2, about 1:1.4, 1:1.6, 1:1.8, or about 1:2, respectively are disclosed. In another embodiment of the CR tablet formulation, compositions containing a ratio of high viscosity PEO to metal halide of about 10:1 to about 1:0, respectively, preferably a ratio selected from about 5:1, 4:1, 3:1, 2:1 and 1:0, respectively, are disclosed. In another embodiment of the CR tablet formulation, compositions containing a ratio of cellulose to metal halide of about 0:1 to about 1:1.6, respectively, preferably about 1:1.1, 1:1.2, 1:1.3, or 1:1.4, respectively are disclosed. In an aspect of this embodiment, about 5%-15% of total coat weight (relative to total tablet weight) of cellulose acetate+polyethylene glycol mixture is present.

Another embodiment of the CR formulation is realized when an alcohol sugar is optionally present in the formulation. A subembodiment of this aspect of the invention is realized when present the alcohol sugar is selected from mannitol, sucrose, lactose. Another subembodiment of this aspect of the invention is realized when the alcohol sugar is mannitol. Another subembodiment of this aspect of the invention is realized when the controlled release formulation in the presence of mannitol in the first layer releases Compound A at a strength of up to 12 mg over a period from about 12-24 hours after placing the composition into an in vivo environment.

Another embodiment of the CR formulation is realized when it comprises additional excipients as needed to ensure the manufacturability, stability or in-vivo performance of the finished dosage form. In an embodiment, 0%-2% lubricating agents such as magnesium stearate, stearic acid, hydrogenated vegetable oil, mineral oil, sodium stearyl fumarate, preferably magnesium stearate is present.

When preparing the tablet, the second layer may contain a lake or dye to enable visual differentiation between the tablet layers as a means to ensure the orifice is drilled in the correct face of the coated tablet. Examples of useful colorants are Red Iron Oxide. Yellow Iron Oxide, Black Iron Oxide and/or FD&C Blue No 2 lake.

The CR coated tablet formulation described above can be further coated with a film coating for the purpose of modifying the appearance or color of the formulation, but without further modifying the rate of release of active ingredient from the formulation. The film coat can be composed of hydroxyproply methylcellulose, hydroxypropyl cellulose, polyvinyl alcohol and/or polyvinyl alcohol—polyethylene glycol copolymer along with plasticizers, opacifiers and/or colorants.

An aspect of this CR formulation is realized when it is dosed at high doses, including starting doses as high as 48 mg, up to about 80 mg with moderate titration. Another aspect of this CR formulation is realized when it is dosed at high doses up to about 80 mg without titration. Another subembodiment of this aspect of the disclosure is CR formulations of Compound A that can be administered once daily at a clinical dose range of about 17 mg to about 47 mg, preferably about 20 mg to about 36 mg, about 20 mg to about 24 mg and more preferably 24 mg without dystonia.

### Definitions

Unless defined otherwise, all technical and scientific terms used herein have the same meaning as commonly understood by one of ordinary skill in the art. Generally, the nomenclature used herein and the laboratory procedures in polymer chemistry, pharmaceutical science, drug delivery, pharmaceutical process technology and pharmacokinetics are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

All ranges disclosed herein are inclusive of the recited endpoint and independently combinable (for example, the range of “from 50 mg to 500 mg” is inclusive of the endpoints, 50 mg and 500 mg, and all the intermediate values). The endpoints of the ranges and any values disclosed herein are not limited to the precise range or value; they are sufficiently imprecise to include values approximating these ranges and/or values.

As used herein, the term “comprising” may include the embodiments “consisting of” and “consisting essentially of.” The terms “comprise(s),” “include(s),” “having,” “has,” “may,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated components, which allows the presence of only the named components or compounds, along with any acceptable carriers or fluids, and excludes other components or compounds.

By “pharmaceutically active agent,” “active ingredient,” “medicament,” or “beneficial agent” is meant Compound A, and pharmaceutically acceptable salts thereof, and derivatives that produce similar localized or systemic effect or effects in animals. Derivatives of the active ingredient, such as esters, ethers and amides without regard to their ionization and solubility characteristics can be used alone or mixed with other compounds. Also, prodrugs of the active agent can be used in a form that, upon release from the tablet, is converted by enzymes, hydrolyzed by body pH or converted by other metabolic processes, to the original form, or to a biologically active form. That is, prodrugs are specifically included within the definition of pharmaceutically active ingredients. Included within the scope of the present invention compositions are those having racemic mixtures and separated enantiomers of the active ingredient. Furthermore, hydrates as well as anhydrous compositions and polymorphs of the active ingredient may be included in compositions of the present invention.

The term “pharmaceutically acceptable salts” means non-toxic salts of the active ingredients which are generally prepared by reacting the free base with a suitable organic or inorganic acid. Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, mandelate, mesylate, methylbromide, methylnitrate, methylsulfate, mucate, napsylate, nitrate, oleate, oxalate, pamaote, palmitate, panthothenate, phosphate/diphosphate, polygalacturonate, salicylate, stearate, subacetate, succinate, tannate, tartrate, teoclate, tosylate, triethiodide, valerate.

As used throughout the specification and appended claims, the following definitions and abbreviations apply:

About: As used herein, the term “about,” when used herein in reference to a value, refers to a value that is the same as or, in context, is plus or minus 10% of the referenced value, e.g. “about 5” means “4.5-5.5”. In general, those skilled in the art, familiar with the context, will appreciate the absolute amount and/or relative degree of difference encompassed by “about” in that context.

Administration: As used herein, the term “administration” refers to the act of providing an active agent, composition, or formulation to a subject. Exemplary routes of administration to the human body can be through the eyes (ophthalmic), mouth (oral), skin (transdermal), nose (nasal), lungs (inhalant), rectal, vaginal, oral mucosa (buccal), ear, by injection (e.g., intravenously (IV), subcutaneously, intratumorally, intraperitoneally, intramuscularly (IM), intradermally (ID) etc.) and the like.

Agent: As used herein, the term “agent” refers to a particle, compound, molecule, or entity of any chemical class including, for example, a small molecule, or a combination or complex thereof. In some embodiments, the term “agent” can refer to a compound, molecule, or entity that includes a polymer, or a plurality thereof.

API: As used herein, the term “API” refers to an active pharmaceutical ingredient, e.g., PDE10 inhibitor, which is a component of the compositions or formulations disclosed herein that is biologically active and confers a therapeutic or prophylactic benefit to a person or animal in need thereof. As used herein, an API is Compound A as the active ingredient.

Biocompatible: As used herein, the term “biocompatible” refers to a material that is not toxic to the body, is pharmaceutically acceptable, is not carcinogenic.

Controlled-release(CR): As used herein, “controlled release” refers to a dosage form which is engineered to release an active agent or other type of substance into the body at a particular rate as a function of time. The rate of release of the pharmaceutically active ingredient from the device (capsule, pill, tablet, etc.) to the environment of use is not immediate, but rather, follows a predetermined pattern. Thus, relatively constant or predictably varying amounts of the beneficial agent can be delivered over a specified period of time.

Dose: As used herein, the term “dose” means a quantity of an agent, API, formulation, or pharmaceutical composition administered or recommended to be administered at a particular time.

Mitigate: As used herein, the term “mitigate,” or “mitigated” means to make less severe, or to reduce.

Patient: As used herein, the term “patient” refers to any human being that is to receive the pharmaceutical compositions, described herein.

Pharmaceutically acceptable: As used herein, the term “pharmaceutically acceptable” refers to excipients (vehicles, additives) and compositions that can reasonably be administered to a subject to provide an effective dose of the active ingredient employed and that are “generally regarded as safe” e.g., that are physiologically tolerable and do not typically produce an allergic or similar untoward reaction, such as gastric upset and the like, when administered to a human. In another embodiment, this term refers to molecular entities and compositions approved by a regulatory agency of the federal or a state government or listed in the U.S. Pharmacopeia or another generally recognized pharmacopeia for use in animals, and more particularly in humans.

Pharmaceutical composition: As used herein, the term “pharmaceutical composition,” refers to a composition containing an active pharmaceutical or biological ingredient, along with one or more additional components, e.g., a composition in which an active agent is formulated together with one or more pharmaceutically acceptable carriers. As used herein, the terms “pharmaceutical formulation” and “formulation” are used interchangeably with “pharmaceutical composition.” In some embodiments, the active agent is present in a unit dose amount appropriate for administration in a therapeutic regimen that shows a statistically significant probability of achieving a predetermined therapeutic effect when administered to a relevant population. Additional components that may be included as appropriate include pharmaceutically acceptable excipients, additives, diluents, buffers, sugars, amino acids, chelating agents, surfactants, polyols, polymers, bulking agents, osmotic agents, stabilizers, lyo-protectants, solubilizers, emulsifiers, salts, adjuvants, tonicity enhancing agents, delivery vehicles, and anti-microbial preservatives. The pharmaceutical compositions or formulations are nontoxic to recipients at the dosages and concentrations employed.

Steady State: the time at which the rate of drug input is equal to the rate of drug elimination (i.e., in a state of equilibrium).

Subject: As used herein, the term “subject,” also referred to as “participant, or “patient,” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Therapeutically Effective Amount: As used herein, the term “therapeutically effective amount” refers to an amount of the active ingredient (e.g., small molecule) sufficient to produce the desired therapeutic effect in a human or animal, e.g., the amount necessary to elicit treat, cure, prevent, or inhibit development and progression of a disease or the symptoms thereof and/or the amount necessary to ameliorate symptoms or cause regression of a disease. Therapeutically effective amount may vary depending on the structure and potency of the active ingredient and the contemplated mode of administration. One of skill in the art can readily determine a therapeutically effective amount of a given molecule.

Viscosity: As used herein, viscosity refers to the measure of a substance's resistance to deformation or flow at a given rate. Viscosity can be measured, for example, by using a viscometer at a given shear rate or shear rates that are appropriately selected by those skilled in the art to be relevant to the viscosity range of the sample of interest.

Volume %, or % by volume: As used herein, the terms “volume %” or “% by volume” refers to parts by volume per hundred parts total volume of polymer carrier, e.g., microparticle, microsphere, or minitab. Unless otherwise indicated to the contrary, percentages (%) reported herein are by volume.

Weight % or % by weight: As used herein, the terms “weight %” or “% by weight” refers to parts by weight per hundred parts total weight of volume of carrier, e.g., blend, carrier, powder, particle, granulation. For example, 10 wt. % active agent would mean 10 parts active agent by weight and 90 parts carrier by weight.

The following examples illustrate the preparation of the drug in the delivery device (e.g., tablet, capsule, etc.) of this invention and the controlled release of active ingredient Compound A into an environment of use and are not to be considered as limiting the invention set forth in the claims appended hereto.

The dosage regimen utilizing the compositions of the present invention is selected in accordance with a variety of factors including type, species, age, weight, sex and medical condition of the patient; the severity of the condition to be treated; the route of administration; the renal and hepatic function of the patient; and the particular active ingredient or salt thereof employed. An ordinarily skilled physician or veterinarian can readily determine and prescribe the effective amount of the drug required to prevent, counter, or arrest the progress of the condition.

Another aspect of the disclosure is CR formulations of Compound A that can be administered at doses as high as 80 mg with good tolerability whereas the immediate-release formulation of Compound A was not escalated above 6 mg (Study 6) due to dystonia adverse events and neuropsychiatric adverse events. Conversely, the safety and tolerability data following administration of Compound A at 80 mg in the CR formulation of the present disclosure did not result in adverse events precluding further dose escalation.

### EXAMPLES

In the following examples, Compound A is the active ingredient. Compositions were prepared using the following ingredients and amounts and formulation procedures.

### Example 1—Immediate Release Formulation

| Ingredient | Unit Formula (mg) |
|---|---|
| Compound A | 1.000 |
| Cellulose, Microcrystalline | 57.27 |
| Dibasic Calcium Phosphate Anhydrous | 114.5 |
| Croscarmellose Sodium | 5.400 |
| Magnesium Stearate | 1.800 |
| Size 3 Hard Gelatin Capsule | |
| TOTAL: | 180.0 |

The immediate-release formulation in Example 1 was prepared as follows: Compound A was triturated with a portion of the dibasic calcium phosphate. This triturate was sequentially blended with a portion of the microcrystalline cellulose, and then with the remaining dibasic calcium phosphate and microcrystalline cellulose and the croscarmellose sodium; this blend was further blended with magnesium stearate to lubricate. The lubricated blend was filled into capsules.

### Example 2—Controlled Release Formulation

| Tablet Core Ingredients | % (weight) | |
|---|---|---|
| Example 2 | Unit Formula (mg) | %(weight) |
| Layer 1 | | |
| Compound A | 12.00 | 6 |
| Polyethylene Oxide, WSR N80 | 145.5 | 72.5 |
| LEO | | |
| Mannitol 200 SD | 41.50 | 21 |
| Magnesium stearate | 1.000 | 0.5 |
| Total 1^{st} Layer Weight | 200.0 | 100 |
| Layer 2 | | |
| Polyethylene Oxide, WSR | 129.6 | 65 |
| Coagulant LEO | | |
| Colorant, FD&C Blue No. 2 (indigo | 0.600 | 0.3 |
| Carmine, E132) Aluminum Lake | | |
| Cellulose, microcrystalline, PH- | 28.80 | 14.2 |
| 200 | | |
| Sodium Chloride | 40.00 | 20 |
| Magnesium Stearate | 1.000 | 0.5 |
| Total 2^{nd} Layer Weight | 200.0 | 100 |
| Total Core Tablet Weight | 400.0 | |
| Coating - | (mg) | |
| Polyethylene Glycol 3350 | 4.900 | 10 |
| Cellulose Acetate CA-398-10 | 44.10 | 90 |
| Coat Weight | 49.00 | 100 |
| Total | 449.0 | |

### Example 3—Controlled Release Formulation (Lower Core Tablet Weight (100 mg Per Layer))

| Tablet Core Ingredients | % (weight) | |
|---|---|---|
| Example 3 | Unit Formula (mg) | %(weight) |
| Layer 1 | | |
| mpound A | 2.00 | 2 |
| Polyethylene Oxide, WSR N80 | 76 | 76 |
| LEO | | |
| Mannitol 200 SD | 21.50 | 21.5 |
| Magnesium stearate | 0.5 | 0.5 |
| Total 1^{st} Layer Weight | 100.0 | 100 |
| Layer 2 | | |
| Polyethylene Oxide, WSR | 64.8 | 64.8 |
| Coagulant LEO | | |
| Colorant, FD&C Blue No. 2 (indigo | 0.300 | 0.3 |
| Carmine, E132) Aluminum Lake | | |
| Cellulose, microcrystalline, PH- | 14.40 | 14.4 |
| 200 | | |
| Sodium Chloride | 20.00 | 20 |
| Magnesium Stearate | 0.500 | 0.5 |
| Total 2^{nd} Layer Weight | 100.0 | 100 |
| Total Core Tablet Weight | 200.0 | |
| Coating | (mg) | |
| Polyethylene Glycol 3350 | 4.400 | 10 |
| Cellulose Acetate CA-398-10 | 39.60 | 90 |
| Coat Weight | 44.00 | 100 |
| Total | 244.0 | |

The CR tablet formulations in Examples 2 through 3 below were prepared as follows: Layer 1, Compound A, the low molecular weight PEO and mannitol were preblended, further blended with a portion of the magnesium stearate, roller-compacted, milled and blended with the remaining magnesium stearate. In Layer 2, the high molecular weight PEO, microcrystalline cellulose, sodium chloride, colorant and magnesium stearate were blended. Layer 1 and Layer 2 blends were compressed to form bilayer tablets. The cellulose acetate and polyethylene glycol were dissolved in an acetone:water (31:1 ratio) solvent system and coated onto the bilayer tablet cores using a perforated-pan tablet coater until the target coating weight was achieved. The coated tablets were drilled with a single hole on the Layer 1 face of each tablet with a laser drill. The tablets were dried at 40° C. to remove residual acetone. The bilayer tablets can consist of two layers of equal weights, or layer weights may be up to 2-fold different, for example from 100:200 mg to 200:100 mg. Total tablet weight may vary from 200-600 mg. Total coat weight can be from 25-75 mg coat weight per tablet, depending on the size and surface area of the core tablet and the permeability of the coat, which can be modulated by the quantity of PEG included in the coat.

Controlled release formulations given by Example 2 and Example 3 exhibited an increase in unit ejection force (values exceeding approximately 1.0-1.5 N/mm²) during bilayer tablet compression where the level of Compound A in the Layer 1 granulation exceeded 6%. Such higher unit ejection forces are undesirable in tablet compression. Unexpectedly, removing mannitol from Layer 1 and replacing with low molecular weight PEO, as shown in Example 4 below, mitigated this effect, resulting in unit ejection forces consistently below 1 N/mm², even at Compound A levels up to approximately 25% in the Layer 1 granulation.

### Example 4

| Tablet Core Ingredients | Unit Formula | |
|---|---|---|
| Example 4 | (mg) | %(weight) |
| Layer 1 | | |
| Compound A | 24.00 | 12 |
| Polyethylene Oxide, WSR N80 | 175 | 87.5 |
| LEO | | |
| Magnesium stearate | 1.000 | 0.5 |
| Total 1^{st} Layer weight | 200.0 | 100 |
| Layer 2 | | |
| Polyethylene Oxide, WSR | 129.6 | 65 |
| Coagulant LEO | | |
| Colorant, FD&C Blue No. 2 (indigo | 0.6000 | 0.3 |
| Carmine, E132) Aluminum Lake | | |
| Cellulose, microcrystalline, PH- | 28.80 | 14.2 |
| 200 | | |
| Sodium Chloride Powder | 40.0 | 20 |
| Magnesium Stearate | 1.00 | 0.5 |
| Total 2^{nd} Layer Weight | 200.0 | 100% |
| Grand Total | 400.0 | |
| Coating | (mg) | |
| Polyethylene Glycol 3350 | 4.900 | 10 |
| Cellulose Acetate CA-398-10 | 44.10 | 90 |
| Coat Weight | 49.00 | 100 |
| Total | 449.0 | |

The controlled release formulation of Example 4 was prepared as follows: Compound A was passed through a #20 mesh screen to delump, low molecular weight PEO was passed through a screening mill (e.g., U20 Comil) fitted with a suitably-sized screen (e.g., 7C075R) operating a approximately 1000 rpm the layer 1 ingredients were then blended in a 3 cu.ft. bin blender for 195 revolutions. Half of the Layer 1 charge of magnesium stearate was screened through a #60 mesh screen to delump and added to the blender with the other Layer 1 ingredients and blended for 90 revolutions to lubricate. The Layer 1 blend was granulated using a roller compactor (e.g., Gerteis TG87) fitted with knurled rolls and a 1 mm square mesh granulator and operated at a roll speed of approximately 2-4 rpm, a roll force of approximately 4-5 kN/cm and a gap of 2 mm. The remaining Layer 1 magnesium stearate was screened through a #60 mesh screen to delump and charged to the 3 cu.ft. bin blender with the Layer 1 granulation and blended for 90 revolutions to lubricate. The Layer 2 excipients, except for magnesium stearate, was screened through a #30 mesh screen to delump. The Layer 2 excipients were blended in a 3 cu.ft. bin blender for 65 revolutions. The blend was passed through a screening mill (e.g., U20 Comil) fitted with a suitably-sized screen (e.g., 7C075R) and operated a approximately 1000 rpm. The materials were further blended in a 3 cu.ft. bin blender for 130 revolutions. The magnesium stearate was passed through a #60 mesh screen to delump, added to the blender with the other Layer 2 excipients and blended for 90 revolutions to lubricate. The Layer 1 and Layer 2 lubricated blends were compressed using a suitable multi-layer tablet press fitted with ⅜ in round standard convex compression tooling targeting layer weights of 200 mg each and a tablet thickness of approximately 5.7 mm. An excess of coating solution containing cellulose acetate:PEG 3350 in a 9:1 ratio in an acetone:water solvent system in a 31:1 ratio and at a solids loading of approximately 5% w/w was prepared, by dispersing the PEG 3350 in the water, combining with the acetone and slowly adding the celulose acetate while stirring to dissolve. The tablet cores were coated with cellulose acetate:PEG 3350 to a target weight gain of 49 mg/tablet using a suitable pan coater operating at an inlet air temperature of approximately 40 C, an outlet temperature of approximately 25 C, a solution spray rate of approximately 45-60 g/minute per spray gun and a gun-to-bed distance of approximately 4 in. A 1 mm diameter and 0.25 mm deep hole was drilled in the center of the Layer 1 face of each tablet using a suitable laser drill (e.g., CMS TT15). The drilled tablets were tray-dried for approximately 36 hrs at 40 C using a suitable drying oven to remove excess acetone.

CR formulations of Compound A were developed and evaluated to determine if sufficient EO could be maintained using once-daily dosing with a slower-rise, more sustained, pharmacokinetic profile. Relative to the IR formulation in Study 6 (Table 1), in Study 4, a single dose study evaluating the PK and tolerability of Compound A, the CR formulation exhibited a delayed Tmax (10-24 h), shallower PTR (~1.3) and consequently a sustained EO between peak and trough (Table 2).

Compound A CR formulations were further evaluated in three multiple-dose studies with treatment durations of 7-18 days in adult participants with schizophrenia and in two of these three studies, healthy non-elderly participants were also evaluated. Study 1 was a randomized, placebo-controlled titration study in which participants (healthy participants (n=10—Table 3) and participants with schizophrenia (n=14—Table 4)) were titrated up from doses of 2 mg to 12 mg as monotherapy or from 2 or 4 mg up to 16 mg (n=19) as adjunctive therapy (participants with schizophrenia only) to prescribed antipsychotic therapy.

Study 2 was designed similarly to Study 1; participants were administered CR Release formulations of Compound A/placebo as monotherapy in participants with schizophrenia (n=16) and healthy participants (n=16), or as adjunctive therapy (n=17) (schizophrenia participants only) and titrated from 4 mg to 24 mg (see Table 3 for illustration of PK at the 24 mg dose level as monotherapy in healthy adults). In Study 2 an additional panel of participants with schizophrenia was administered CR formulations of Compound A/placebo as monotherapy and titrated from 8 mg to 48 mg (n=26)(see Table 4 for illustration of PK at the 48 mg dose level).

In Study 3, one panel of participants [(n=8)—monotherapy in adult participants with schizophrenia] were titrated from 16 mg to 24 mg or placebo, while in another panel participants (n=18) received 24 mg/placebo without titration (Table 5) to determine if titration was required for tolerability.

Overall, CR formulations of Compound A were generally well-tolerated in schizophrenia participants as monotherapy and as adjunctive therapy across Studies 1 through 3 and dose titrations. In Study 1 (Table 4), dystonia was reported in 5 of 33 (15%) schizophrenia participants receiving CR formulations of Compound A (3 following monotherapy [1 each following 2 mg, 4 mg and 12 mg] and 2 following adjunctive therapy [8 mg, 16 mg]). All events responded immediately to benztropine.

In Study 2, when doses were titrated from 4 mg to 24 mg and from 8 mg to 48 mg, no Compound A-related dystonia was observed. Cmax values of ~1800 nM and C24 values of ~1200 nM were achieved at the highest dose level in Study 2 (Table 4), substantially higher than the 376 nM Cmax associated with dystonia in healthy participants in Study 6 (IR formulation-Table 1).

In Study 3, Compound A was generally well tolerated when dosing was initiated at 16 mg (data not shown in Table 5) or 24 mg with no titration, with PK estimates similar to what had been observed previously for the CR formulation (see Table 5 for PK of the 24 mg dose without titration). There were 2 events of transient dystonia following the first dose of 16 mg, but both events responded to treatment and participants continued dosing without recurrence. No events of dystonia were reported when dosing was initiated at 24 mg without titration (Table 5). Other adverse events (i.e., depression, anxiety and stress) across all three studies were mostly mild or moderate in intensity and there was no clear dose-related increase in specific events (Table 6).

Unexpectedly across Study 1 and Study 2, in healthy participants, tolerability was improved at doses up to about 47 mg. Moreover, in Study 2 participants with schizophrenia did not experience dystonia at doses from about 4 mg to 48 mg, although maximum and trough concentration (Cmax and C24) values, and calculated EO at Cmax and C24 (Table 4) were similar or greater than those associated with dystonia in the study with the IR formulation (Table 1). Similar trends of sustained exposures and occupancy estimates were also observed in participants with schizophrenia administered Compound A as monotherapy in Study 1 and Study 2 (Table 4). Exposures of CR formulation of Compound A and sustained EO estimates in participants with schizophrenia administered as adjunctive therapy were similar to participants receiving monotherapy.

Overall, studies 1-3 also showed in both healthy participants and participants with schizophrenia tolerability was improved as doses from about 17 mg to about 47 mg did not result in dystonia. Additionally, there were no reports of dystonia following administration of the CR formulation up to 12 mg (n=9) and 24 mg (n=12) in healthy participants.

In another study, Study 4 (a single dose study evaluating the slowest intermediate and fastest CR formulations), all subjects received a single dose of 2 mg Compound A four times (4 treatment periods) under fasted conditions where treatment was provided after an 8-hour overnight fast and fasting continued up to 4 hours after dosing. The observed Tmax range was 6-16 hours, 10-24 hours and 20-24 hours for the fastest, intermediate and slowest-releasing CR formulations, respectively, administered under fasted condition. The PTR for the intermediate CR formulation was ~1.3 (Table 2).

In another study, Study 5 (no table illustration), across approximately 54 participants that received CR formulation of Compound A 48 mg/placebo on Day 1 and 60 mg/placebo on Day 2, or 48 mg/placebo on Day 1 and 80 mg/placebo on Day 2 or 48 mg/placebo on Day 1 and on Day 2 and 80 mg on Day 3, only 2 participants reported dystonia following 48 mg of compound A. Overall, with the CR formulation over a dose range of 20 mg to 80 mg, 2 of ~82 (2.4%) participants reported compound A related dystonia which is significantly less than that seen with the IR formulation (Note: preliminary study results).

In a study of an IR formulation of Compound A (Study 6) where PK and tolerability were evaluated (Table 1), while no healthy participants reported dystonia following the 1 mg dose, following the 3 mg dose (n=12) and 6 mg dose (n=6), 3 participants (25%) and 1 participant (17%) reported dystonia, respectively. Following the 3 mg dose, dystonia began at 1 hour 10 min, 1 hour 27 min and 1 hour 31 minutes post dose. Following the 6 mg dose dystonia began at 3 hour 50 min post dose. This participant also reported depressive feelings and nightmares. Another subject also reported depressive feelings, anxiety and stress. Due to these adverse events it was decided not to dose escalate further.

Based on the EO-concentration relationship, Compound A CR doses of 24 mg and higher are expected to produce sustained EO of about, 65%, 77%, or 80% or higher at steady-state, which is greater than the EO associated with in the IR formulation. Surprisingly, the high levels of sustained EO achieved by the CR formulation were not associated with an increase in dystonia. No events of dystonia were reported in the healthy participants in Study 1 and Study 2, and in participants with schizophrenia (across studies 1, 2, 3, and 5), dystonia was observed with a much lower incidence-about 2% (2 participants of ~82) at doses ranging from 20 mg to 80 mg. Even more surprising is no events of dystonia were reported with the high levels of sustained EO achieved by the CR formulation at doses ranging from 17 mg to 47 mg in both healthy participants and participants with schizophrenia. These findings suggest that high sustained EO can be achieved by the CR formulation of Compound A, with an unexpectedly favorable tolerability profile with regard to dystonia.

In another healthy subject study, Study 7 (no table illustration), healthy subjects were dosed from 8 mg/placebo to 72 mg/placebo (n=12), no dystonia was reported up to and including doses of 48 mg. Plasma exposures in this study were generally similar those observed in the other multiple dose studies reported here (data not shown).

TABLE 1
PK Summary Statistics and Calculated % EO of
IR Formulation in Healthy Adults (Study 6)

| | AUC0- | | | | | Cmax | C24 |
|---|---|---|---|---|---|---|---|
| Dose | inf^{a} | Cmax | Tmax | C24 | | EO | EO |
| (mg) | (nM*hr) | (nM)^{a} | (hr)^{b} | (nM)^{a} | PTR | (%) | (%) |
| 1 | 1020 | 141 | 1.50 | 8.06 | 17.5 | 40 | 3.6 |
| | (46) | (30) | | (120) | | | |
| 3 | 2800 | 376 | 0.875 | 21.3 | 17.6 | 64 | 8.9 |
| | (27) | (17) | | (91) | | | |
| 6 | 6490 | 759 | 1.13 | 59.8 | 12.7 | 78 | 21 |
| | (33) | (18) | | (74) | | | |

^{a}Values represent geometric mean (% CV)
^{b}Values represent median time
PTR: Peak-to-Trough concentration ratio
EO: Enzyme Occupancy

TABLE 2
PK Summary Statistics and Calculated % EO of
CR Formulation in Healthy Adults (Study 4)

| | AUC0- | | | | | Cmax | C24 |
|---|---|---|---|---|---|---|---|
| Dose | inf^{a} | Cmax | Tmax | C24 | | EO | EO |
| (mg) | (nM*hr) | (nM)^{a} | (hr)^{b} | (nM)^{a} | PTR | (%) | (%) |
| 2 | 1280 | 56.7 | 20.00 | 43.7 | 1.3 | 21 | 17 |
| | (32) | (33) | | (38) | | | |

^{a}Values represent geometric mean (% CV)
^{b}Values represent median time
PTR: Peak-to-Trough concentration ratio
EO: Enzyme Occupancy

TABLE 3
PK Summary Statistics and Calculated %
EO of CR Formulation in Healthy Adults

| | | | AUC0- | | | | | Cmax | C24 |
|---|---|---|---|---|---|---|---|---|---|
| | Dose | | 24^{a} | Cmax | Tmax | C24 | | EO | EO |
| Study | (mg) | Day | (nM*hr) | (nM)^{a} | (hr)^{b} | (nM)^{a} | PTR | (%) | (%) |
| 1 | 12 | 14 | 9820 | 519 | 11.00 | 375 | 1.38 | 71 | 63 |
| | | | (21.9) | (14.2) | | (28.0) | | | |
| 2 | 24 | 16 | 15600 | 807 | 13.99 | 625 | 1.29 | 79 | 74 |
| | | | (36.1) | (34.0) | | (39.7) | | | |
| | | 18 | 13600 | 741 | 11.99 | 433 | 1.71 | 77 | 62 |
| | | | (59.6) | (48.0) | | (111.3) | | | |

TABLE 4
PK Summary Statistics and Calculated % EO of CR Formulation
as Monotherapy in Adult Participants with Schizophrenia

| | | | AUC0- | | | | | Cmax | C24 |
|---|---|---|---|---|---|---|---|---|---|
| | Dose | | 24^{a} | Cmax | Tmax | C24 | | EO | EO |
| Study | (mg) | Day | (nM*hr) | (nM)^{a} | (hr)^{b} | (nM)^{a} | PTR | (%) | (%) |
| 1 | 12 | 14 | 12300 | 628 | 16 | 459 | 1.37 | 74 | 68 |
| | | | (27.0) | (28.5) | | (33.4) | | | |
| 2 | 24 | 16 | 24600 | 1250 | 10.09 | 949 | 1.32 | 85 | 82 |
| | (Panel B) | | (36.6) | (37.7) | | (45.7) | | | |
| | | 18 | 26600 | 1300 | 12.05 | 1010 | 1.29 | 86 | 83 |
| | | | (38.3) | (36.8) | | (47.5) | | | |
| | 48 | 13 | 31800 | 1670 | 16.02 | 1370 | 1.22 | 88 | 86 |
| | (Panel D) | | (54.5) | (51.9) | | (47.5) | | | |
| | | 15 | 34400 | 1890 | 16.02 | 1160 | 1.63 | 89 | 81 |
| | | | (66.5) | (52.9) | | (97.8) | | | |

^{a}Values represent geometric mean (% CV)
^{b}Values represent median time
PTR: Peak-to-Trough concentration ratio
EO: Enzyme Occupancy

TABLE 5
PK Summary Statistics and Calculated % EO of CR
Formulation as Monotherapy without Titration in
Adult Participants with Schizophrenia (Study 3)

| | | AUC0- | | | | | Cmax | C24 |
|---|---|---|---|---|---|---|---|---|
| Dose | | 24^{a} | Cmax | Tmax | C24 | | EO | EO |
| (mg) | Day | (nM*hr) | (nM)^{a} | (h)^{b} | (nM)^{a} | PTR | (%) | (%) |
| 24 | 7 | 19500 | 1110 | 11.03 | 677 | 1.63 | 83 | 75 |
| | | (48.1) | (46.7) | | (52.2) | | | |

^{a}Values represent geometric mean (% CV)
^{b}Values represent median time
PTR: Peak-to-Trough concentration ratio
EO: Enzyme Occupancy

TABLE 6
Participants With Non-Serious Related Adverse Events
(Part 1 Panel B: Participants With Schizophrenia - Young Adults)
(Incidence > 0% in One or More Treatment Groups)

| | | | Compound | | | | | |
|---|---|---|---|---|---|---|---|---|
| | Screening | | A 24 mg | | Placebo | | Total | |
| | n | (%) | n | (%) | n | (%) | n | (%) |
| Participants in population | 18 | | 14 | | 4 | | 18 | |
| with one or more Non- | 0 | (0.0) | 5 | (35.7) | 0 | (0.0) | 5 | (27.8) |
| Serious Related | | | | | | | | |
| Adverse Event | | | | | | | | |
| with no Non-Serious | 18 | (100.0) | 9 | (64.3) | 4 | (100.0) | 13 | (72.2) |
| Related Adverse Event | | | | | | | | |
| Investigations | 0 | (0.0) | 1 | (7.1) | 0 | (0.0) | 1 | (5.6) |
| Alanine aminotransferase | 0 | (0.0) | 1 | (7.1) | 0 | (0.0) | 1 | (5.6) |
| increased | | | | | | | | |
| Metabolism and nutrition | 0 | (0.0) | 1 | (7.1) | 0 | (0.0) | 1 | (5.6) |
| disorders | | | | | | | | |
| Decreased appetite | 0 | (0.0) | 1 | (7.1) | 0 | (0.0) | 1 | (5.6) |
| Musculoskeletal and | 0 | (0.0) | 1 | (7.1) | 0 | (0.0) | 1 | (5.6) |
| connective tissue | | | | | | | | |
| disorders | | | | | | | | |
| Musculoskeletal stiffness | 0 | (0.0) | 1 | (7.1) | 0 | (0.0) | 1 | (5.6) |
| Nervous system disorders | 0 | (0.0) | 3 | (21.4) | 0 | (0.0) | 3 | (16.7) |
| Hypertonia | 0 | (0.0) | 1 | (7.1) | 0 | (0.0) | 1 | (5.6) |
| Somnolence | 0 | (0.0) | 2 | (14.3) | 0 | (0.0) | 2 | (11.1) |

| | Post Trial | |
|---|---|---|
| | n | (%) |
| Participants in population | 18 | |
| with one or more Non-Serious | 0 | (0.0) |
| Related Adverse Event | | |
| with no Non-Serious Related | 18 | (100.0) |
| Adverse Event | | |
| Investigations | 0 | (0.0) |
| Alanine aminotransferase increased | 0 | (0.0) |
| Metabolism and nutrition disorders | 0 | (0.0) |
| Decreased appetite | 0 | (0.0) |
| Musculoskeletal and connective | 0 | (0.0) |
| tissue disorders | | |
| Musculoskeletal stiffness | 0 | (0.0) |
| Nervous system disorders | 0 | (0.0) |
| Hypertonia | 0 | (0.0) |
| Somnolence | 0 | (0.0) |

Every participant is counted a single time for each applicable row and column.
Adverse events listed under Post Trial refer to AEs reported after 14 day follow up period post last dose received.
The Total does not include Screening or Post Trial.
Panel B: Titration 24 mg or placebo (Day 1-7).
One participant discontinued prior to the 24 mg dose on Day 2. One participant discontinued after the 24 mg dose on Day 4. One participant discontinued after the 24 mg dose on Day 3. One participant discontinued prior to the 24 mg dose on Day 5.
Adverse event terms are from MedDRA Version 25.
Dystonia was not observed in any patient with the 24 mg dose in the study.

1. A controlled-release tablet formulation comprising a:
1) first layer having about 1%-25% by weight of pharmaceutically acceptable Compound A, represented by structural formula I: 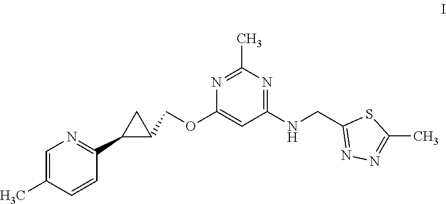
or a pharmaceutically acceptable salt, ester derivative, geometric isomer, stereoisomer, or optical isomer thereof and about 60-90% by weight low viscosity polyethylene oxide (PEO) polymer;
2) second layer comprising about 50%-70% by weight high viscosity polyethylene oxide polymer, about 10%-40% by weight metal halide and about 5%-30% by weight tableting agent; and
3) semipermeable coating wherein the coating comprises about 1%-15% by weight of the tablet, said tablet having an orifice in the first layer, wherein said formulation induces no dystonia after administration.

2. The formulation according to claim 1, wherein the low viscosity polyethylene oxide has a molecular weight range (g/mol) of about 100,000 to 300,000 and is present in the first layer at about 65%-88% by weight.

3. The formulation of claim 1, wherein the high viscosity polyethylene oxide has a molecular weight range (g/mol) of about 4,000,000 to 7,000,000 and is present in the second layer at about 50%-70% by weight.

4. The formulation of claim 1, wherein the tableting agent is selected from lactose, spray dried lactose, microcrystalline cellulose, mannitol, sorbitol, dibasic calcium phosphate dehydrate, and calcium sulphate dehydrate.

5. The formulation of claim 4, wherein the tableting agent is microcrystalline cellulose, present at about 10%-20% by weight.

6. The formulation of claim 1, wherein the metal halide is selected from sodium chloride, potassium chloride, calcium chloride, and potassium iodide, or a mixture thereof, and is present at about 15%-25% by weight.

7. The formulation of claim 1, wherein the semipermeable coating comprises cellulose acetate, ethyl cellulose, or a combination thereof and low molecular weight polyethylene glycol.

8. The formulation of claim 1, wherein Compound A, or a pharmaceutically acceptable salt, ester derivative, geometric isomer, stereoisomer, or optical isomer thereof is present at about 4%-25% by weight, and low viscosity polyethylene oxide has a molecular weight range (g/mol) of about 100,000 to 300,000 and is present at about 65%-88% by weight of total first layer weight.

9. The formulation of claim 1, wherein
a) the high viscosity polyethylene oxide has a molecular weight range (g/mol) of about 4,000,000 to 7,000,000 and is present at about 60%-68% by weight,
b) the tableting agent is present up to 10%-20% by weight, and
c) the metal halide is present at about 15%-25% by weight
of total weight of the second layer.

10. The formulation of claim 1, wherein 5%-15% of the semipermeable coating comprises cellulose acetate, ethyl cellulose, or mixture thereof, wherein the cellulose acetate, ethyl cellulose, or mixture thereof contains about 5%-20%, by weight, of low molecular weight polyethylene glycol.

11. The formulation of claim 1, further comprising a sugar alcohol selected from mannitol, sucrose, and lactose.

12. The formulation of claim 1, that exhibits peak to trough concentration levels of Compound A from about 1.0 to about 3.5.

13. The formulation of claim 1, comprising from about 17 mg to about 47 mg of Compound A which can be administered once daily with no incidence of dystonia.

14. The formulation of claim 1, comprising Compound A at doses at 16 mg or higher which exhibits sustained muscarinic enzyme occupancy at both peak and trough of about 65% or higher.

15. The formulation of claim 14, wherein when administered in a patient population suffering from schizophrenia there are no reported incidents of dystonia.

16. A method for treating schizophrenia or other psychiatric disorders in a patient in need thereof, comprising administering to the patient a controlled-release formulation of Compound A according to claim 1 at doses of 16 mg or higher without inducing dystonia in the patient.
